(19) United States
(12) Patent Application Publication
(10) Pub. No.: US 2023/0143153 A1
(43) Pub. Date: May 11, 2023

(54) HATS WITH SOUND DIRECTING ASSEMBLIES

(71) Applicant: Versi LLC, Sheridan, WY (US)

(72) Inventor: Daniel C. Buck, Sheridan, WY (US)

(73) Assignee: Versi LLC, Sheridan, WY (US)

(21) Appl. No.: 17/977,512

(22) Filed: Oct. 31, 2022

Related U.S. Application Data
(60) Provisional application No. 63/276,193, filed on Nov. 5, 2021.

(51) Int. Cl. G10K 11/22; A42B 1/245

(52) U.S. Cl. CPC G10K 11/22; A42B 1/245; G10K 11/08

(57) ABSTRACT

An apparatus worn on a head of the user. The apparatus includes a hat having a front, an opposite back, and a crown position at the front. A sound directing assembly has a body coupled to the crown of the hat. The body of the sound directing assembly is configured to receive sound. A sound tube extends from the body to an earpiece that is configured to be inserted into an ear of the user. The sound tube is configured to direct the sound received by the body to the earpiece such that the user can hear the sound.

### CROSS REFERENCE TO RELATED APPLICATION

This application claims the benefit of U.S. Provisional Patent Application No. 63/276,193, filed Nov. 5, 2021, which is incorporated herein by reference in its entirety.

### FIELD

The present disclosure relates to hats, and specifically to hats with sound directing assemblies that direct sound to the ears of the user.

### BACKGROUND

Hearing loss is a common problem around the world, which arises from excessive noise, from aging, from disease, and heredity-based factors. People with hearing loss may have difficulty engaging in conversation with friends and family. Hearing loss may also make it difficult to enjoy hobbies and activities such as attending a concert, a sporting event, or other gatherings of people. Likewise, hearing loss may make it challenging to communicate with professionals such as physicians, to decipher audible instructions, or to recognize alarms, warnings, or doorbells.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

Certain embodiments according to the present disclosure generally relate to an apparatus worn on a head of the user. The apparatus includes a hat having a front, an opposite back, and a crown position at the front. A sound directing assembly has a body coupled to the crown of the hat. The body of the sound directing assembly is configured to receive sound. A sound tube extends from the body to an earpiece that is configured to be inserted into an ear of the user. The sound tube is configured to direct the sound received by the body to the earpiece such that the user can hear the sound.

Another embodiment generally relates to a method for making an apparatus for assisting hearing for a user. The method includes providing a hat having a front, an opposite back, and a crown position at the front and providing a sound directing assembly having a body configured to receive the sound, an earpiece configured to be inserted into an ear of the user, and a sound tube that extends between the body and the earpiece. The method further includes coupling the body of the sound directly assembly to the crown of the hat such that the sound tube directs the sound received by the body to the earpiece to assist the user in hearing the sound.

Various other features, objects, and advantages will be made apparent from the following description taken together with the drawings.

### BRIEF DESCRIPTION OF THE DRAWINGS

The present disclosure is described with reference to the following Figures. The same numbers are used throughout the Figures to reference like features and like components.

FIG. 1 is a front view of an exemplary apparatus of the present disclosure on the head of a user.

FIG. 2 is a side view of the apparatus of FIG. 1.

FIG. 3 is a side view of another exemplary apparatus of the present disclosure.

FIG. 4 is a partial top view of another exemplary apparatus of the present disclosure.

FIG. 5 is an enlarged view of an exemplary bracket and an exemplary earpiece.

FIG. 6 is a sectional top view of an exemplary sound directing apparatus of the present disclosure.

FIG. 7 is an isometric side view another exemplary sound directing apparatus of the present disclosure.

FIG. 8 is a front view of another exemplary apparatus of the present disclosure on the head of a user.

### DETAILED DESCRIPTION

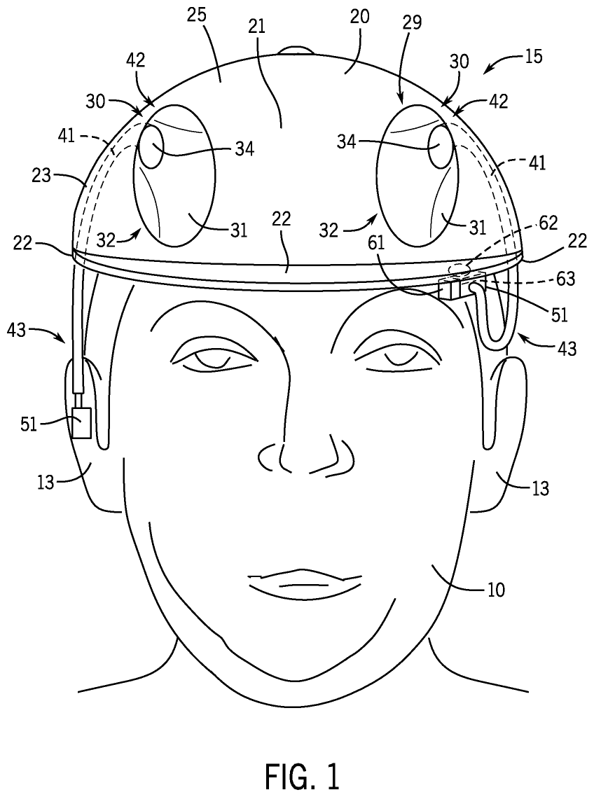

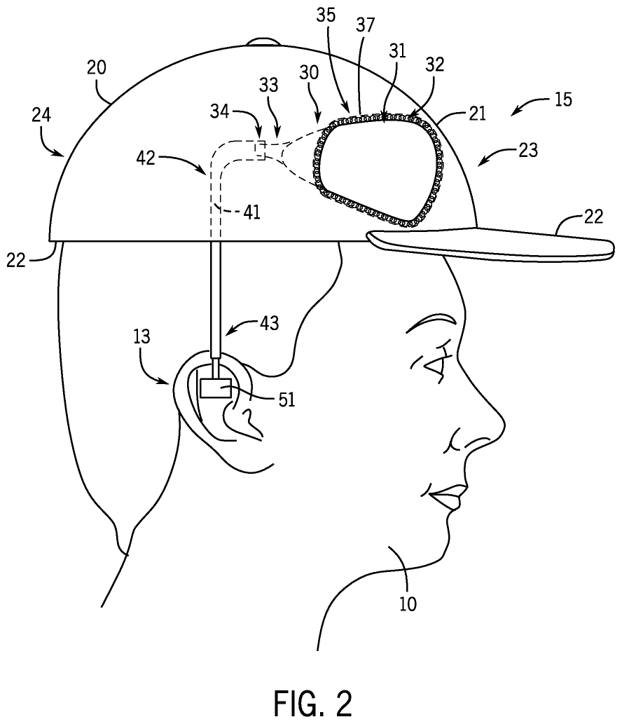

FIGS. 1-2 depict an exemplary apparatus 15 of the present disclosure on the head of a user. The apparatus 15 includes a hat 20, such as a baseball cap, and one or a pair of sound directing assemblies 30 that are configured to receive sound and direct the sound to the ears 13 of the user. In this manner, the apparatus 15 and the assemblies 30 help the user hear sounds that may otherwise be too faint to hear by collecting, directing, and concentrating the sound towards the ears 13.

The hat 20 includes a crown 21 positioned at a front 23 of the hat 20 and a brim 22 that extends along at least a portion of the perimeter of the hat 20. In one example, the brim 22 extends along the entire perimeter of the hat 20 including along the front 23 and an opposite back 24 of the hat 20. The crown 21 includes an exterior surface 25 that is viewable by third parties and an opposite interior surface (not depicted) that faces the head 10 of the user.

One or more assemblies 30 are coupled to the hat 20, whereby the embodiment of FIGS. 1 and 2 has a pair of assemblies 30. Each of the assemblies 30 includes a body 31 that receives sounds and a sound tube 41 connected to the body 31. The sounds received by the bodies 31 travel through the sound tubes 41 to corresponding earpieces 51. Each of these components is described in further detail hereinbelow.

Each body 31 is coupled to the crown 21 of the hat 20. The body 31 may be coupled to the exterior surface 25 and/or the interior surface of the crown 21. In certain embodiments, an opening 29 is provided through the crown 21 and the body 31 is at least partially aligned with the opening 29 so as to avoid attenuation of the sound entering the body 31 through the crown 21.

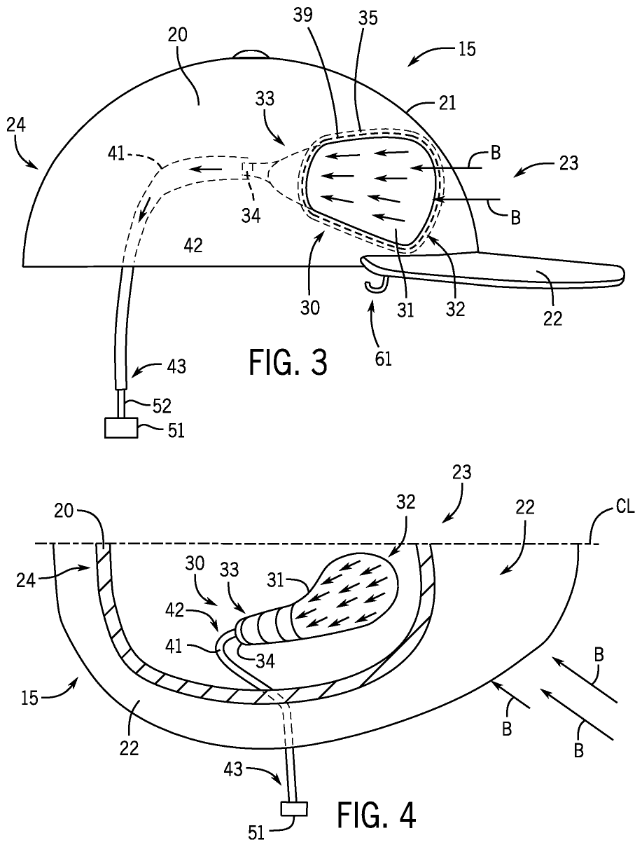

In the embodiment of FIGS. 1 and 2, the body 31 of each assembly 30 is generally parabolically shaped such that the body 31 has an enlarged first body end 32 and an opposite narrow second body end 33. As such, the body 31 funnels the sound received via the first body end 32 to the second body end 33 and the sound passes through a port 34 at the second body end 33. That is, the body 31 concentrates and directs the sounds received via the first body end 32 to the second body end 33. The body 31 is coupled to the exterior surface 25 and/or the interior surface (not depicted) of the crown 21 of the hat 20 in any suitable matter. For example, the body 31 may be coupled to the interior surface of the crown 21 via adhesives 37 (FIG. 2) or stitches 39 (FIG. 3), or may use hook and loop fasteners, staples, rivets, or other known coupling techniques. The body 31 may also or alternatively be sandwiched between two layers of materials that form the crown 21.

The body 31 can be coupled to the crown 21 in any position. It may be advantageous to position one or more of the bodies 31 such that the first body end 31 faces the direction of the sound the user wishes to hear. In one exemplary embodiment (see FIG. 1), the two assemblies 30 are coupled to the hat 20 such that the first body end 32 of one of the bodies 31 faces in a direction extending away from the front-left portion of the user's head 10 and the first body end 32 of the other body 31 faces in a direction extending away from the front-right portion of the user's head 10. Accordingly, in this example the sound received by each body 31 is directed to the respective ears of the user (e.g., the body 31 at the front-left directs sound to the left ear) such that the assemblies 30 give the user stereo-like sound and three-dimensional locality.

The sound tube 41 can be formed from any suitable material (e.g., plastic, rubber), and preferably the sound tube 41 is flexible so as to conform to the shape of the user and prevent discomfort. A first tube end 42 of the sound tube 41 is connected to the second body end 32, and an opposite second tube end 43 of the sound tube 41 is connected to the earpiece 51. The first tube end 42 and/or second tube end 43 of the sound tube 41 may be connected to the second body end 32 and the earpiece 51 in a variety of manners. By way of example, the sound tube 41 may be integrally formed with the body 31 and/or the earpiece 51, may incorporate a press-fit arrangement (e.g., a barbed end at the second body end 32 received within an interior of a resilient sound tube 41), or may be clamps to secure the connections.

The present inventors have recognized that in certain configurations it is advantageous for at least the connection between the second tube end 43 of the sound tube 41 and the earpiece 51 to be easily connected and disconnected. This allows for replacement of the earpiece 51 to customize the appropriate size for a user, or to replace over time due to wear or for hygienic purposes. The connection between the second tube end 42 of the sound tube 41 and the second body end 32 of the body 30 may also advantageously be easily connected and disconnected to replace the sound tube 41. The sound tube 41 may need to be replaced due to damage or becoming less compliant over time, or to adjust the length of the sound tube 41 for the user's needs.

With continued reference to FIGS. 1 and 2, the sound tube 41 is capable of allowing the sound to freely pass therethrough. Therefore, the sound tube 41 directs the sound from the body 31 to the earpiece 51. The sound tube 41 is positioned between the exterior surface 25 of the hat 20 and the head of the user. In one specific example, the sound tube 41 is coupled to the interior surface of the hat 20 with stitching or glue. In another example, the sound tube 41 is sandwiched between two fabric layers of the hat 20. This technique of sandwiching may be advantageous for increasing the durability of the apparatus, whereby the sound tube 41 is subsequently shielded from damage on both the exterior surface 25 of the hat and from contacting the head of the user at the inner surface. The sandwiching design may also be more comfortable for the user to make the sound tube 41 less discernable against the user's head.

The earpiece 51 receives the sound from the sound tube 41 and dispenses the sound to the ear 13 of the user. The earpiece 51 can include rubber and/or foam that maintain the earpiece in the ear of the user. The earpiece 51 is designed to be inserted into the concha and/or the ear canal of the ear 13 and retained therein during use. The earpiece 51 may include a filter (not shown) that allows the sound to pass but prevents debris and moisture from passing into the earpiece 51.

Referring specifically to FIG. 1, a bracket 61 is connected to the brim 22 of the hat 20, such as via adhesives, stitches, rivets, or other techniques known in the art. The bracket 61 is configured for retaining the earpiece 51 on the hat 20, rather than in the user's ear 13, when the earpiece 51 is not in use. For instance, the user may selectively move the earpiece 51 and/or a portion of the sound tube 41 into engagement with the bracket 61. This holds the earpiece 51 so as to not fall off the apparatus 15 and/or dangle uncontrollably alongside the user's head. The bracket 61 can be fixed relative to the brim 22. In other examples, the bracket 61 includes one or more operable or movable components such that bracket 61 is movable into and between an extended position in which the earpiece 51 can be coupled to the bracket 61 and a retracted position next to the brim 22.

Additional embodiments of apparatuses 15 according to the present disclosure are provided herein. It should be noted that these apparatuses 15 and/or assemblies 30 may include one or more of the features and/or components of the apparatuses 15 and/or the assemblies 30 described with reference to FIGS. 1-2. Similarly, it should be understood that any of the features and/or components of the example apparatuses 15 and/or assemblies 30 described below can be combined with the apparatuses 15 and/or the assemblies 30 described with reference to FIGS. 1-2.

Referring now to FIG. 3, another example apparatus 15 of the present disclosure is depicted. The sound tube 41 is shown connected between the body 31 and the earpiece 51. It should be noted that an inner diameter of the first tube end 42 of the sound tube 41 is greater than an outer diameter of the port 34 at the second body end 33 of the body 31 such that the first tube end 42 slides onto the port 34. As such, the first tube end 42 and the port 34 form a “male-female” connection and compression forces between the port 34 and the first tube end 42 maintain the connection therebetween. Similarly, the inner diameter of the second tube end 43 is greater than the outer diameter of a male end 52 of the earpiece 51 such that the second tube end 43 slides onto the male end 52. Compression forces between the male end 52 and the second tube end 42 maintain the connection therebetween.

FIG. 4 depicts another example apparatus 15 of the present disclosure. One side or hemisphere of the hat 20 is depicted, cut at a centerline CL between the left and right sides of the hat 20. The crown 21 (see FIG. 1) is excluded to expose the assembly 30. In this example, the first body end 32 receives sounds from the front-right side of the user (see sound arrows B).

FIG. 5 depicts an enlarged view of an example bracket 61. The bracket 61 is coupled to the brim 22 and extends vertically downward from the brim 22. By way of example, the bracket 61 may retain the earpiece 51 in position below the brim 22 via being shaped as a hook (see FIG. 3), being shaped so as to pinch the sound tube 41, or may retain the earpiece 51 via corresponding components 62, 63, such as pieces of hook and loop fasteners or magnets (see FIG. 1).

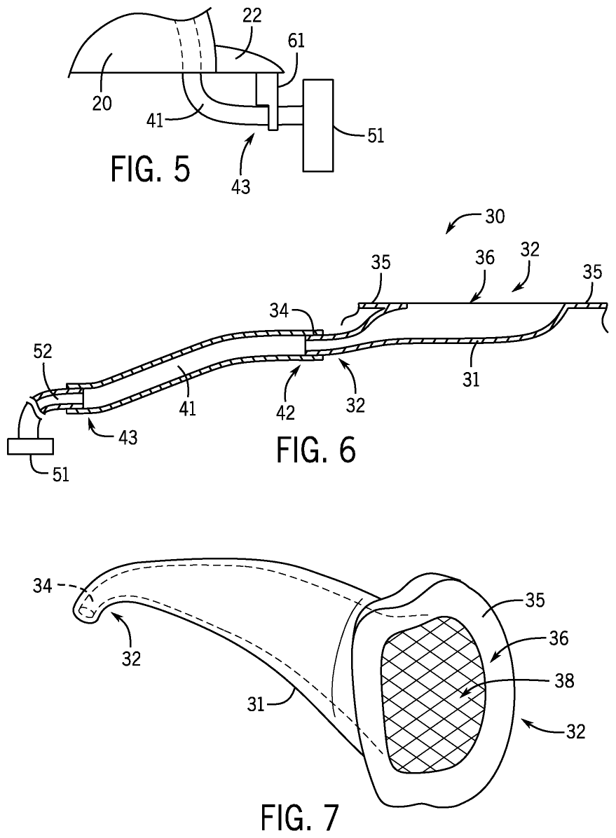

FIG. 6 depicts an example top view of an example assembly 30, sectioned to show an interior of the body 31 and sound tube 41. In this example, the body 31 includes a flange 35 that extends around the perimeter of the first body end 32 and defines an opening 36 at the first body end 32. The opening 36 receives the sound, and in certain examples, the opening 36 can be covered by a cover 38, such as a porous fabric, that extends there across (see FIG. 7). The flange 35 may serve as a basis for coupling the body 31 to the crown 21, in particular providing a surface for an adhesive or for stitching the body 31 to the crown 21 therethrough.

FIG. 7 depicts a side view of an example assembly 30, which shows that the opening 36 at the first body end 32 of the body 31 may be directed so as to face not only forward or sideways from the perspective of the user when coupled to the hat 20. Rather, the opening 36 may face an oblique direction that is both forward and sideways, as discussed above. In certain embodiments, as shown in FIG. 7, the opening 36 is formed within an edge at the first body end 32 that is non-planar. In this manner, the opening 36 faces multiple angles relative to the user rather than simply being formed as a planar area that is oblique to the front of the hat 20.

The opening 36 may again be covered by fabric or another material for aesthetic purposes and/or to prevent moisture or debris from entering the body 31. The cover 38 may cover only the opening 36, the entire flange 35, or any combination of the two. In certain examples it is advantageous for the cover 38 to be made of a material that causes less sound attenuation that the material of the crown 21, as it is desirable to maximize the amplitude of the sound being received within the body 31 and funneled to the user. In certain cases, this may include a cover 38 that has the same material composition as the crown 21, but a lesser thickness. The cover 38 may have the same appearance as the crown 21 so as to obscure the presence thereof, may provide an aesthetically-appealing contrast to the crown 21, or other configurations for hiding or accentuating the presence of the body 31.

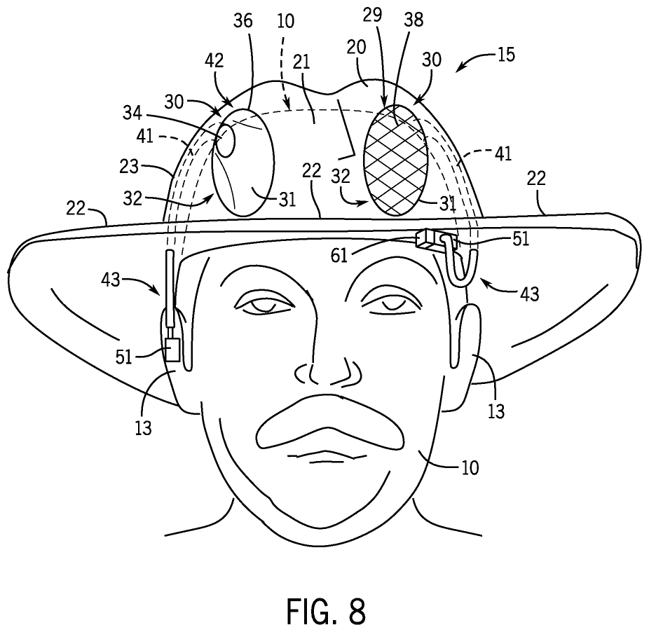

FIG. 8 depicts another embodiment of a hat 20 having two assemblies 30 according to the present disclosure. In this example, the opening 36 in the body 31 for one assembly 30 is shown to have a cover 38, whereas the other is exposed to show inside from the front view. In the example shown, the opening 36 extends partially above the head 10 of the user when the hat 20 is worn. It should be recognized that the entire opening 36 may alternatively be positioned at or below the top of the head, above the head, or in other positions entirely. By way of example, both earpieces 51 may be connected via sound tubes 41 to a single body 31, which may be positioned as shown in FIG. 8, in a single central location, and/or in other positions. It should further be recognized that just as multiple earpieces 51 may be connected to a single body 31, a single earpiece 51 may be connected to multiple bodies. In this manner, the presently disclosed apparatus provide for customized configurations based on the needs of the user (e.g., multiple bodies 31 feeding into a single ear having hearing loss) and/or the context for use (e.g., forward-only facing openings 36 in bodies 31 for attending performances and events in which all intended noise is at the front, with undesirable background noise coming from the side).

The present disclosure further relates to methods for making for an apparatus for assisting hearing for a user. In certain examples, the method includes providing a hat having a front, an opposite back, and a crown position at the front and providing a sound directing assembly having a body configured to receive the sound, an earpiece configured to be inserted into an ear of the user, and a sound tube that extends between the body and the earpiece. The method further includes coupling the body of the sound directly assembly to the crown of the hat such that the sound tube directs the sound received by the body to the earpiece to assist the user in hearing the sound.

In certain embodiments, the sound enters the body at a first end thereof and the body is coupled to the crown such the body is inside the hat. In other embodiments, the sound enters the body at a first end thereof and the method further includes creating an opening that extends through the crown, whereby the body is coupled to the crown such that the first end of the body is aligned with the opening through the crown so as to avoid attenuation of the sound passing through the crown. Certain methods further include covering the first end body with a material different than a material of the crown, as discussed above.

The present inventors have recognized that the apparatuses and methods disclosed herein advantageously provide a hands-free, aesthetically pleasing solution for people affected by hearing loss. These solutions avoid the technical challenges associated with electronic hearing assisting devices (e.g., electric hearing aids), do not require a power source, and can be made more cost-effectively than products currently known in the art. Moreover, the hats may be lighter and/or more durable due to the lack of electrics and power sources, making the experience more comfortable for the user.

In the present description, certain terms have been used for brevity, clarity, and understanding. No unnecessary limitations are to be inferred therefrom beyond the requirement of the prior art because such terms are used for descriptive purposes and are intended to be broadly construed. The different apparatuses, systems, and method steps described herein may be used alone or in combination with other apparatuses, systems, and methods. It is to be expected that various equivalents, alternatives and modifications are possible within the scope of the appended claims.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An apparatus worn on a head of the user, the apparatus comprising:
a hat having a front, an opposite back, and a crown position at the front; and
a sound directing assembly having a body coupled to the crown, the body configured to receive sound and the sound directing assembly further comprising a sound tube that extends from the body to an earpiece that is configured to be inserted into an ear of the user;
wherein the sound tube is configured to direct the sound received by the body to the earpiece such that the user can hear the sound.

2. The apparatus according to claim 1, wherein the hat has a brim, and further comprising a bracket that selectively retains the earpiece on the hat.

3. The apparatus according to claim 1, wherein the sound enters the body at a first end thereof, and wherein the first end is positioned inside of the crown.

4. The apparatus according to claim 1, wherein the sound enters the body at a first end thereof, further comprising a flange positioned at the first end of the body, wherein the body is coupled to the crown via the flange.

5. The apparatus according to claim 1, wherein the body is coupled to the crown via an adhesive.

6. The apparatus according to claim 1, wherein the sound enters the body at a first end thereof, wherein an opening extends through the crown, and wherein the first end of the body is aligned with the opening through the crown so as to avoid attenuation of the sound passing through the crown.

7. The apparatus according to claim 1, wherein the sound enters the body at a first end thereof, and wherein at least a portion of the first end is positioned obliquely to the front of the hat.

8. The apparatus according to claim 1, wherein an edge at a first end of the body defines an area through which the sound enters the body, and wherein the area is non-planar.

9. The apparatus according to claim 1, wherein the sound enters the body at a first end thereof, wherein an opening extends through the crown, and wherein the first end of the body is aligned with the opening through the crown, further comprising a cover that covers the first end of the body.

10. The apparatus according to claim 9, wherein the cover comprises a material that provides less sound attenuation than a material of the crown.

11. The apparatus according to claim 9, wherein the cover has a thickness that is less than a thickness of the crown.

12. The apparatus according to claim 1, wherein a centerline extends between the front and the back of the hat to bisect the hat into a left side and a right side, and wherein the body is positioned such that the hat extends farther than the body from the centerline.

13. The apparatus according to claim 1, wherein the sound tube is a flexible tube.

14. The apparatus according to claim 1, wherein the body is positioned entirely inside the hat.

15. The apparatus according to claim 1, wherein the sound enters the body at a first end thereof, and wherein the first end is at least partially positioned above the head of the user when the apparatus is worn.

16. A method for making an apparatus for assisting hearing for a user, the method comprising:
providing a hat having a front, an opposite back, and a crown position at the front;
providing a sound directing assembly having a body configured to receive the sound, an earpiece configured to be inserted into an ear of the user, and a sound tube that extends between the body and the earpiece; and
coupling the body of the sound directly assembly to the crown of the hat such that the sound tube directs the sound received by the body to the earpiece to assist the user in hearing the sound.

17. The method according to claim 16, wherein the sound enters the body at a first end thereof, and wherein the body is coupled to the crown such the body is inside the hat.

18. The method according to claim 16, wherein the sound enters the body at a first end thereof, further comprising creating an opening that extends through the crown, and wherein the body is coupled to the crown such that the first end of the body is aligned with the opening through the crown so as to avoid attenuation of the sound passing through the crown.

19. The method according to claim 18, further comprising covering the first end body with a material different than a material of the crown.

20. An apparatus worn on a head of the user, the apparatus comprising:
a hat having a front, an opposite back, and a crown position at the front, wherein an opening extends through the crown;
a sound directing assembly having a body coupled to the hat such that a first end of the body aligns with the opening through the crown, wherein sound passing through the opening is received in the first end of the body, the sound directing assembly further having a flexible sound tube that extends from the body to an earpiece configured to be positioned in or on an ear of the user;
a cover that covers the first end of the body, the cover comprising a material that is different than a material of the crown; and
a bracket that selectively retains the earpiece on the hat;
wherein the sound tube is configured to direct the sound received by the body to the earpiece such that the user can hear the sound.
